(19) United States
(12) Patent Application Publication
(10) Pub. No.: US 2008/0223367 A1
(43) Pub. Date: Sep. 18, 2008

(54) Method and apparatus for treating airway obstruction

(76) Inventors: Brian J. Cox, Laguna Niguel, CA (US); Robert F. Rosenbluth, Laguna Niguel, CA (US); Dean Schaefer, Laguna Hills, CA (US)

Correspondence Address: INSKEEP INTELLECTUAL PROPERTY GROUP, INC, 2281 W. 190TH STREET, SUITE 200, TORRANCE, CA, 90504, US

(21) Appl. No.: 12/011,793

(22) Filed: Jan. 29, 2008

Related U.S. Application Data
(60) Provisional application No. 60/887,035, filed on Jan. 29, 2007.
(60) Provisional application No. 60/888,439, filed on Feb. 6, 2007.
(60) Provisional application No. 60/895,957, filed on Mar. 20, 2007.

(51) Int. Cl. A61M 16/00; A61F 5/56

(52) U.S. Cl. 128/204.18; 128/848

(57) ABSTRACT

Methods and Devices for treating airway openings and breathing disorders including obstructive sleep apnea are disclosed. Structures and methods disclosed herein maintain and preserve airway openings against posterior collapse of the tongue.

### RELATED APPLICATIONS

This application claims priority to U.S. Provisional Application Ser. No. 60/887,035 filed Jan. 29, 2007 entitled Method And Apparatus For Treating Airway Obstruction; U.S. Provisional Application Ser. No. 60/888,439 filed Feb. 6, 2007 entitled Method And Apparatus For Treating Airway Obstruction; and U.S. Provisional Application Ser. No. 60/895,957 filed Mar. 20, 2007 entitled Method And Apparatus For Treating Airway Obstruction, all of which are hereby incorporated by reference.

### BACKGROUND OF THE INVENTION

This invention relates to the treatment of airway openings and breathing disorders including obstructive sleep apnea.

The tongue is a mobile muscular organ that can assume a variety of shapes and positions. The tongue has a relatively fixed inferior part that is attached to the hyoid bone and mandible. The tongue is involved with mastication, taste, articulation, and oral cleansing. Its two main functions are forming words during speaking and squeezing food into the pharynx when swallowing. The forces applied by the tongue in producing movements during speech and swallowing are significantly higher than those causing collapse during apnea.

Obstructive sleep apnea (OSA) is a potentially life-threatening disorder, which affects up to 2-4% of the adult population. OSA is characterized by an intermittent cessation of airflow in the presence of continued inspiratory effort. OSA can be triggered when the base of the tongue relaxes and collapses during sleep. When the tongue collapses, it moves in the posterior direction so that it partly or completely obstructs the airway. When these obstructive episodes occur, an affected person will transiently arouse, regain muscle tone and reopen the airway. Because these arousal episodes typically occur 10 to 60 times per night, sleep fragmentation causes excessive daytime sleepiness. Some patients with OSA experience over 100 transient arousal episodes per hour. Patients with severe OSA have higher risk of systemic and pulmonary hypertension, cardiac arrhythmia and stroke.

Various methods are known in the art of treatment of OSA and have varying degrees of success. A common means of treating sleep apnea is the use of a machine that delivers increased air pressure to the nose and mouth of the sleeper. These machines are described as CPAP (Continuous Positive Airway Pressure) machines. They entail wearing of a mask, headgear, and flexible hose which is attached to the air pump. Although effective, the CPAP machine is not widely accepted by patients. Discomfort, the sound of the air pump, claustrophobia and the stigma of being seen while wearing the mask, headgear, and hose have all been listed as reasons for not continuing use of the CPAP.

Surgical procedures for treatment of OSA include uvulo-pharyngeal-palatoplasty (UPPP), midline glossectomy, hyoid suspension, mandibular advancement and tracheostomy. In UPPP, the lateral portion of the soft palate is removed, a painful procedure with high post-operative morbidity and only partial success. Hyoid suspension is performed only in highly selected cases, generally people with large tongues. Mandibular advancement is essentially facial reconstruction where the jaw is re-aligned by moving both the upper and lower jaws forward. These surgical procedures are complex, invasive, entail considerable morbidity, and exhibit only moderate results. In severe cases of OSA, tracheotomy may be the only currently effective surgical treatment. Patients with a tracheostomy often experience significantly compromised speech due to the associated changes in airflow patterns and social stigma. Thus, while tracheostomy has been almost uniformly effective in relieving OSA, it is used in only a small percentage of patients.

Various oral OSA devices for preventing posterior movement of the tongue have been developed. One type of oral device involves a tongue retention device that advances and secures the tongue using suction, or mechanical tongue depression and stabilization. However, this type of oral device has limited success and is poorly tolerated by the user. A second type of oral device is a mandibular repositioning device that advances the lower jaw relative to the fixed upper jaw to expand the cross-sectional area of the pharynx thereby improving airflow and preventing collapse. These devices have been variably effective, but commonly have both comfort and compliance problems.

Thus, there has been a long felt need for a better treatment of breathing disorders such as OSA that is effective and acceptable to most patients.

### BRIEF DESCRIPTION OF THE DRAWINGS

FIG. 1 is a cutaway view of a patient's mouth and throat area.

FIG. 2 is a cutaway view of a patient's mouth and throat area.

FIG. 3 is a front view of a preferred embodiment of the present invention.

FIG. 4 is a cross-section view of a preferred embodiment of the present invention.

FIG. 5 is a cutaway view of a patient's mouth and throat area with a preferred embodiment of the present invention.

FIG. 6 is a cross-section view of a preferred embodiment of the present invention.

FIG. 7 is a side view of a preferred embodiment of the present invention.

FIG. 8 is a perspective view of a preferred embodiment of the present invention.

FIG. 9 is a cutaway view of a patient's mouth and throat area with a preferred embodiment of the present invention.

FIG. 10 is a cutaway view of a patient's mouth and throat area with a preferred embodiment of the present invention.

FIG. 11 is a cross-section view of a preferred embodiment of the present invention.

FIG. 12 is a cutaway view of a patient's mouth and throat area with a preferred embodiment of the present invention.

FIG. 13 is a perspective view of a preferred embodiment of the present invention.

FIG. 14 is a cross-section view of a preferred embodiment of the present invention.

FIG. 15 is a cross-section view of a preferred embodiment of the present invention.

FIG. 16 is a graph of stress and strain pertaining to a preferred embodiment of the present invention.

FIG. 17 is a cross-section view of a preferred embodiment of the present invention.

FIG. 18 is a cutaway view of a patient's mouth and throat area with a preferred embodiment of the present invention.

FIG. 19 is a cutaway view of a patient's mouth and throat area with a preferred embodiment of the present invention.

FIG. 20 is a cross-section view of a preferred embodiment of the present invention.

FIG. 21 is a perspective view of a preferred embodiment of the present invention.

FIG. 22 is a cross-section view of a preferred embodiment of the present invention.

FIG. 23 is a perspective view of a preferred embodiment of the present invention.

FIG. 24 is a cutaway view of a patient's mouth and throat area with a preferred embodiment of the present invention.

FIG. 25 is a cutaway view of a patient's mouth and throat area with a preferred embodiment of the present invention.

FIG. 26 is a perspective view of a preferred embodiment of the present invention.

FIG. 27 is a cutaway view of a patient's mouth and throat area.

FIG. 28 is a perspective view of a preferred embodiment of the present invention.

FIG. 29 is a perspective view of a preferred embodiment of the present invention.

FIG. 30 is a cross-section view of a preferred embodiment of the present invention.

FIG. 31 is a cross-section view of a preferred embodiment of the present invention.

FIG. 32 is a cross-section view of a preferred embodiment of the present invention.

FIG. 33 is a cross-section view of a preferred embodiment of the present invention.

FIG. 34 is a cross-section view of a preferred embodiment of the present invention.

FIG. 35 is a cross-section view of a preferred embodiment of the present invention.

FIG. 36 is a front view of a preferred embodiment of the present invention.

FIG. 37 is a cross-section view of a preferred embodiment of the present invention.

FIG. 38 is a cross-section view of a preferred embodiment of the present invention.

### DETAILED DESCRIPTION OF THE INVENTION

Methods and apparatus are provided for the treatment of obstructed airways. The devices illustrated in preferred embodiments are particularly useful for the treatment of breathing disorders such as obstructive sleep apnea or OSA by preventing the complete closure of an airway.

### I. First Preferred Embodiments

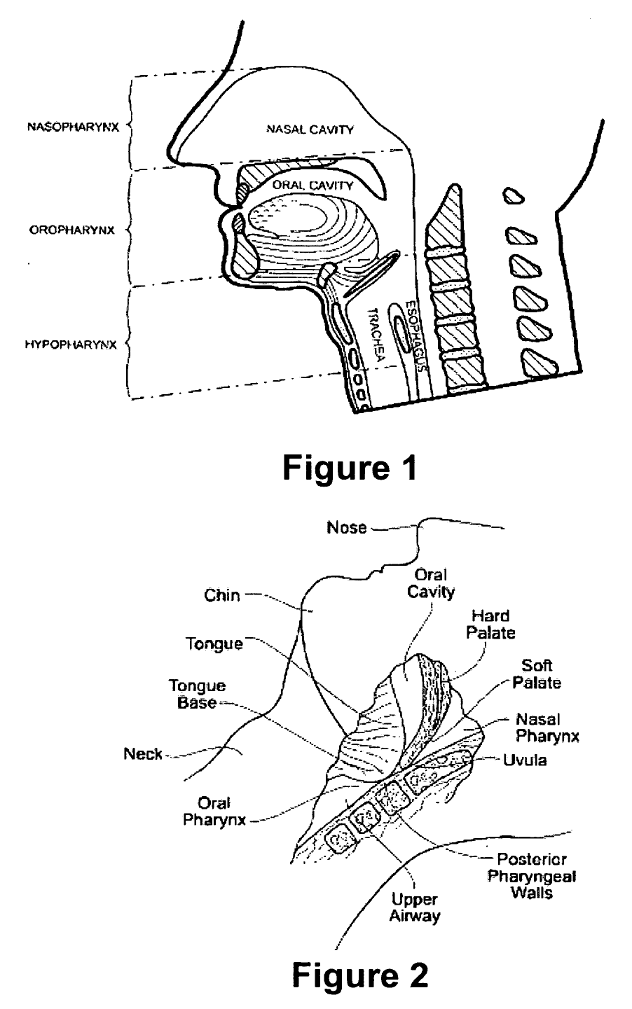

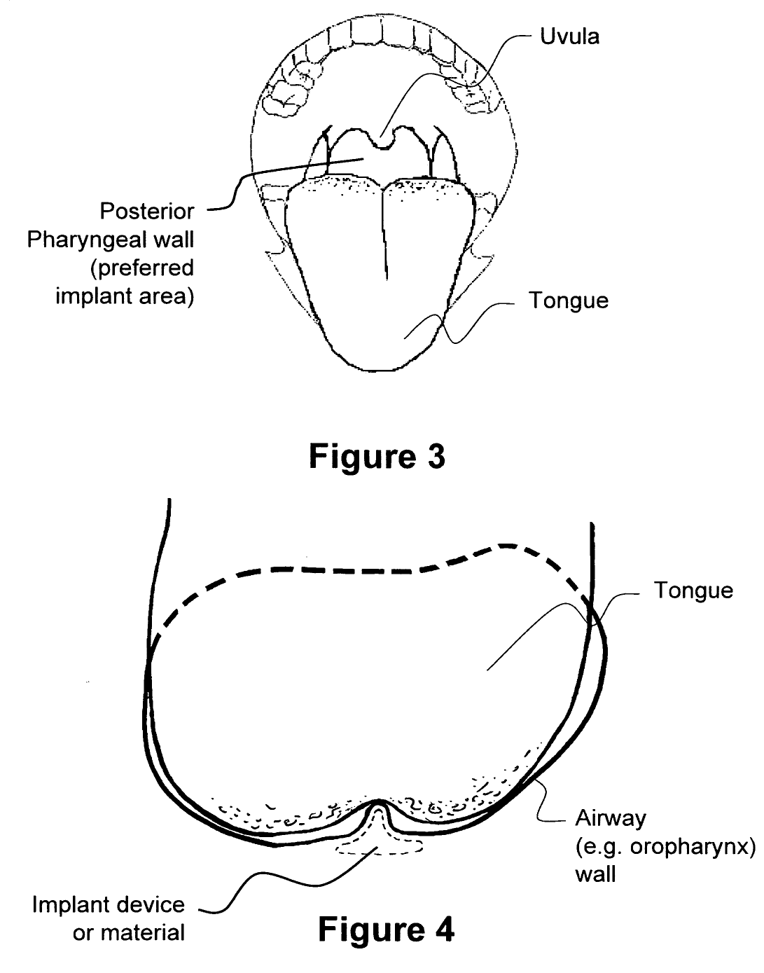

The invention in accordance with the first preferred embodiments comprises a biocompatible material that is placed within or attached to an airway wall to prevent another object or body part from substantially blocking airflow by creating a seal against the airway wall. In the case of OSA, the device prevents the tongue from sealing against the posterior wall of the upper airway called the oropharynx. The oropharynx is shown in FIG. 1. In sleep apnea, the tongue collapses against the posterior wall of the oropharynx as shown in FIG. 2. The device is preferably implanted in the posterior pharyngeal wall as shown in FIG. 3. In one embodiment, the device comprises a material that is injectable or insertable into airway wall to form one or more protrusions into the airway wall. Preferably, the device is placed in the tissue under the airway wall mucosa. Preferably, the protrusion is a longitudinal ridge that is substantially aligned with the axis of the airway as shown in FIG. 4. Alternatively, the protrusion may be an oval and round configuration. When a body part such as the tongue moves against the airway wall in the vicinity of the device, the device prevents complete closure of airway. Thus complete obstruction of air flow is prevented. For the injectable compositions, the material may be fibers, microspheres, microparticles or the like. Exemplary injectable tissue bulking agents are described by Li et al. in U.S. Patent Application 20060251697, by Bourne et al. in U.S. Pat. No. 7,131,997 and by Vogel et al. in U.S. Pat. No. 6,660,301 which are all herein incorporated in their entirety by reference.

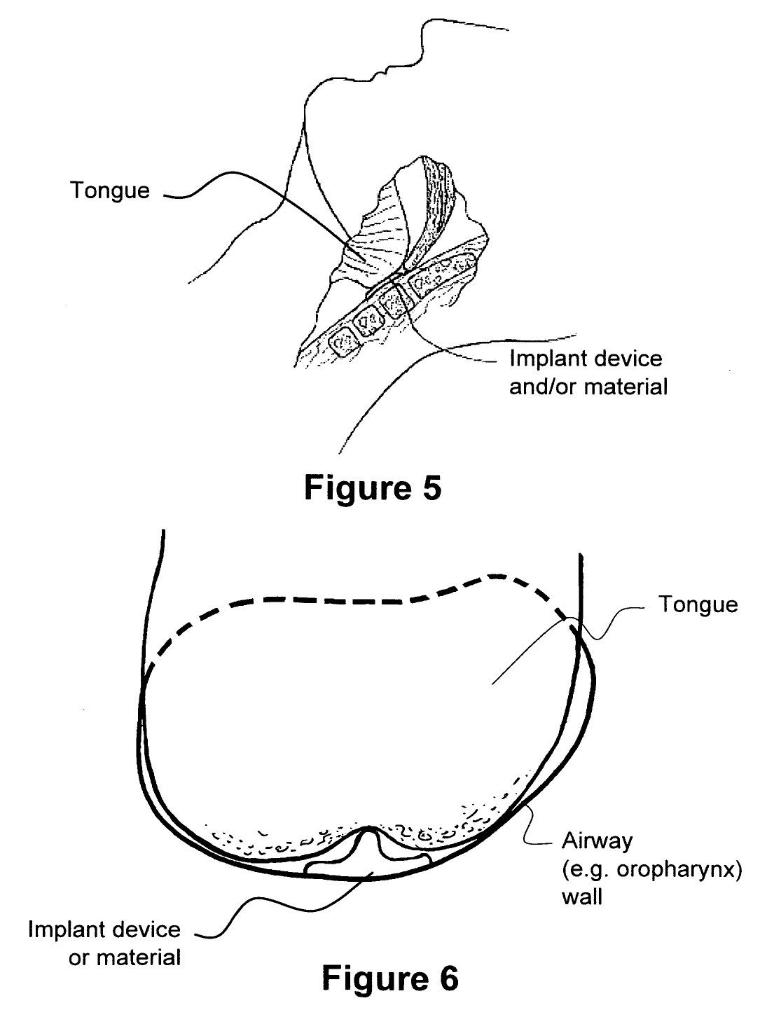

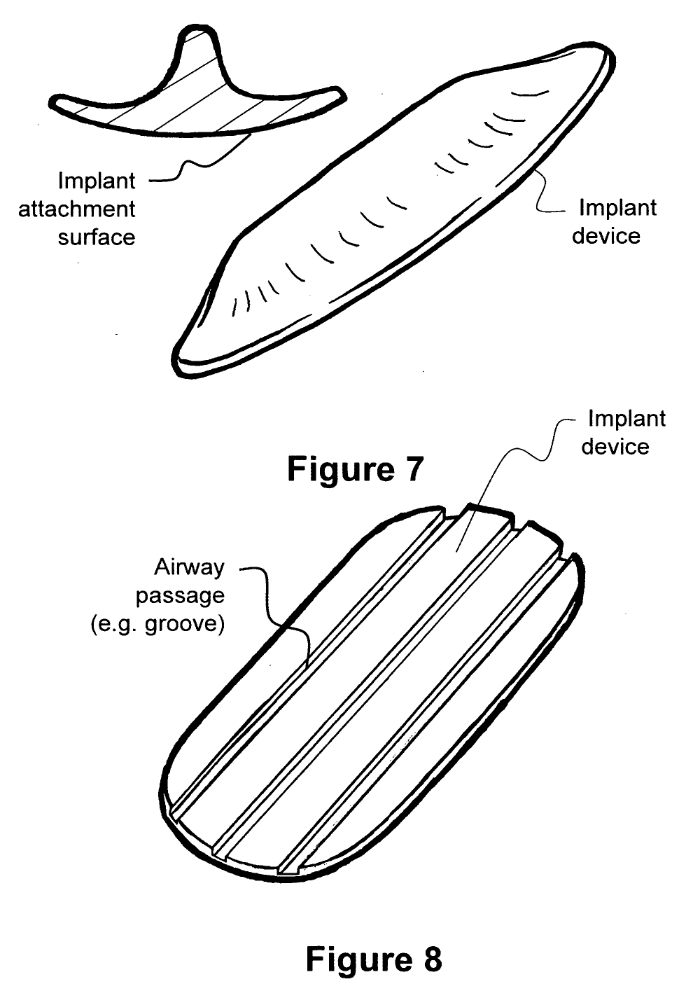

In one embodiment, the device comprises a biocompatible material member that is attached to the inner surface of an airway. In one embodiment, the device may be configured to provide at least one protrusion into the airway. In another embodiment, the device comprises one or more airway passages. The device may comprise a number of shapes including elongate shapes with various cross-sections and a spherical cap. In one embodiment, the attaching member is an elongate shape that can form a substantially longitudinal ridge in the airway as shown in FIGS. 5 and 6. Preferably, the device is placed substantially aligned with the axis of the airway. The attaching member may have a number of cross-sectional shapes including but not limited to square, rectangular, triangular, and semi-circular. Optionally, the device may have flanges for facilitating attachment. Preferably, the attachment surface and/or flange is curved to substantially match the radius of the airway as shown in FIG. 7. The attaching member may be attached to the airway wall by attachment means known in the art of surgery such as adhesives, sutures, clips, hooks, barbs, staples and the like. Preferably, the device is soft and flexible so that it flexes easily with the movements of pharyngeal wall muscles. Optionally, the device may comprise structures to facilitate flexibility. In any of the above embodiments where the device is attached to the airway wall, the device may optionally have one or more recessed passageways or grooves to facilitate the passage of air even when the tongue or other object is pressed against it as shown in FIG. 8. Preferably, the groove(s) are oriented to be substantially aligned with the axis of the airway when implanted. Preferably, the combined cross-sectional area of the groove(s) is between about 1 and 30 square millimeters and more preferably between about 3 and 20 square millimeters.

The device may be made of various biomaterials known in the art of implant devices including but not limited to polymers, metals, and biological materials. Suitable polymers include polyurethanes, silicones, polypropylene, polyvinyl alcohol, polyesters (e.g. polyethylene terephthalate or PET) and PolyEtherEther Ketone (PEEK). Suitable metals include cobalt-chrome alloys, nickel-titanium alloys, platinum, stainless steel, titanium, gold, and tungsten. Optionally, one or more surfaces of the device may be porous (preferably microporous) to facilitate the ingrowth of tissue. In one embodiment, the device comprises a cellular structure such as a foam or sponge-like structure. Optionally, the device may comprise fibers or strands that are woven, braided or knitted as is known in the art of textile vascular grafts. Optionally, the device may be constructed to provide the elution or delivery of one or more beneficial drug(s) and/or other bioactive substances into the airway or into the airway wall and surrounding tissue.

Optionally, in any of the wall-attached embodiments the device may comprise a sensor to measure physiologic parameters such as pressure or air flow. The sensor may communicate with an external device to allow recording of the sensor data. Further, the device may comprise an operable member that allows the device to move in response to change in a measured physiologic parameter.

Also provided are methods of treating breathing disorders such as obstructive sleep apnea. In one embodiment, the method comprises the following steps:
a) Attaching a structure to at least one surface of an airway of a patient, wherein the structure comprises one or more protrusions.
b) Preventing the complete closure of the airway by said protrusion stopping the movement of a body part from sealing against the airway in the vicinity of the device.

In another embodiment, the method comprises the following steps;
a) Inserting or injecting a biocompatible material underneath the mucosa of an airway wall to create one or more protrusions into the airway.
b) Preventing the complete closure of the airway by said protrusion(s) stopping the movement of a body part from sealing against the airway in the vicinity of the device.

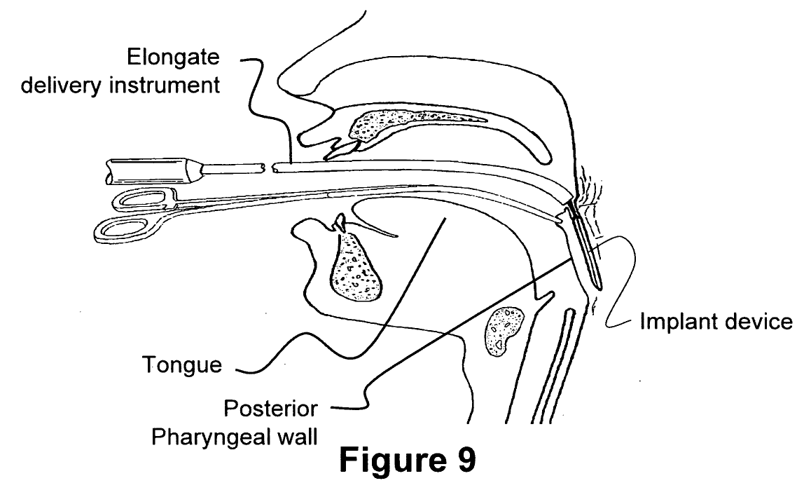

Optionally, the method may further include any of the following steps;
c) Advancing an elongate instrument into the oropharynx via a trans-oral approach as shown in FIG. 9.
d) Attaching the structure to a patient's airway wall using sutures, hooks, barbs, clips, staples and/or a bioadhesive.
e) Attaching a structure to an airway wall wherein the structure comprises one or more air passageways or grooves to facilitate the passage of air when the tongue is pressed against said structure.

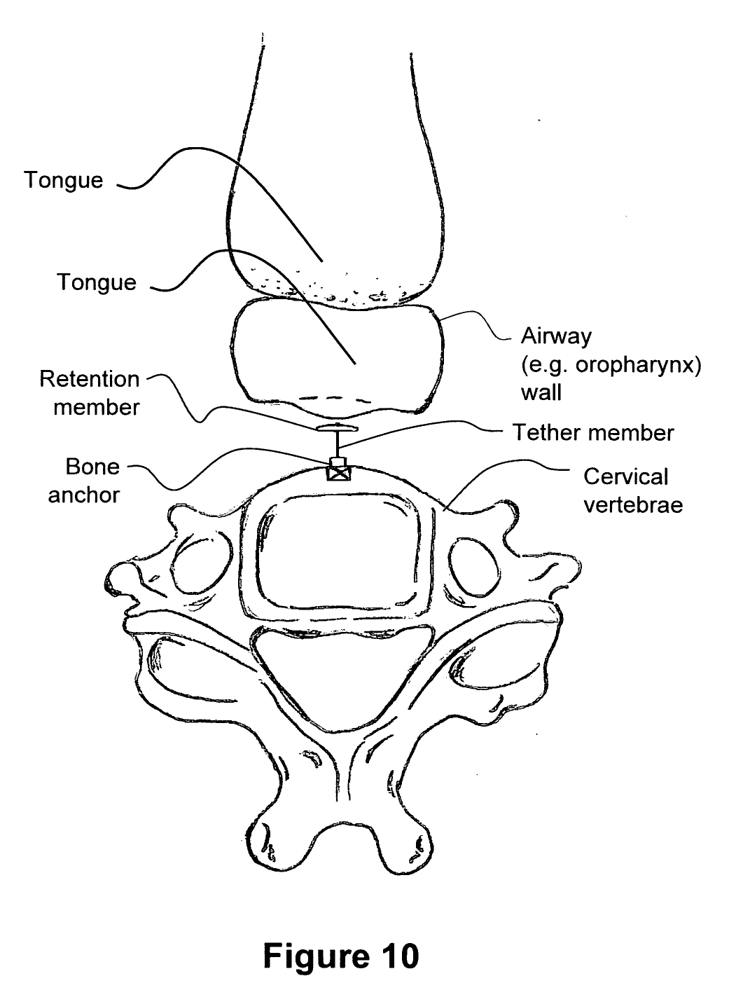

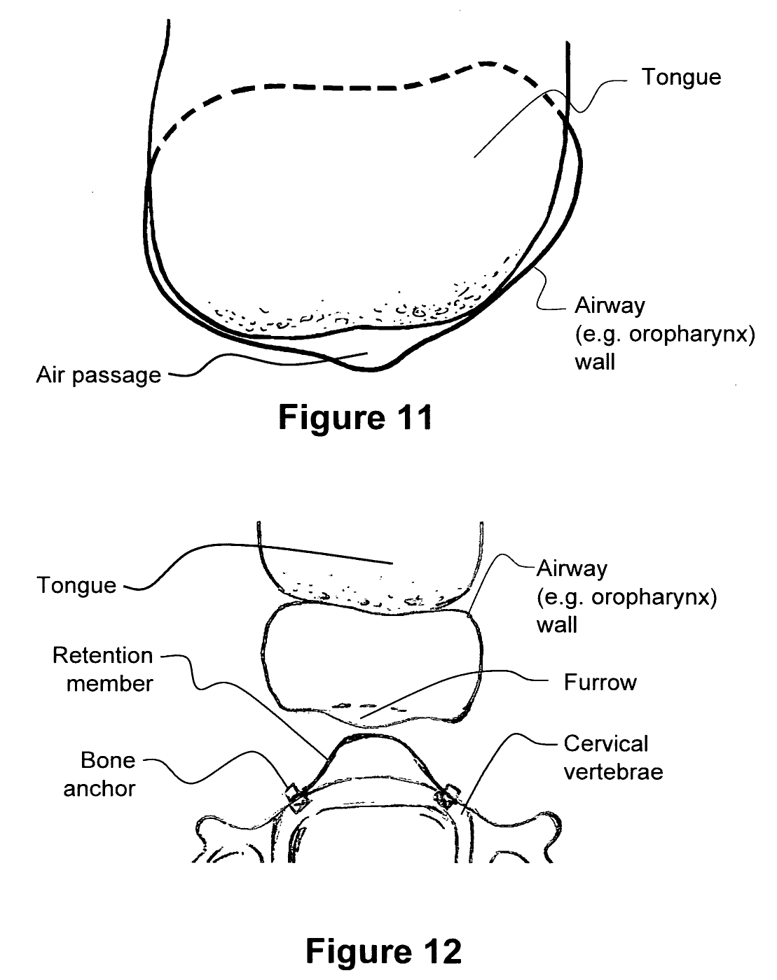

In one embodiment, the airway of a patient is enlarged by implantation of a retention member that pulls at least a portion of the airway wall in the radially outward direction thus expanding the cross-sectional area of the airway. The retention member is attached or anchored to a relatively stable structure such as bone or hard tissue with a tether member such as a suture as shown in FIG. 10. The tether may be connected to bone using one or more bone anchors. Suitable bone anchors are commercially available from Depuy Mitek, Inc. Preferably, the tether member is attached to the third cervical vertebrae. The retention member forms a narrow depression or furrow in the pharyngeal wall such that when a body part (e.g. tongue) moves against the wall, the depression or furrow provides an air passage thus preventing a complete seal of the airway as shown in FIG. 11. In another embodiment the retention member may form a substantially U-shaped member that extends radially from the airway. Similar to a suspension procedure that is done to support the urinary tract, a strip member of biocompatible material may be placed in a looping fashion around a portion of the pharyngeal wall muscles and then connected to an adjacent anatomical structure such as a cervical vertebrae as shown in FIG. 12. Preferably, at least two anchors are used to attach the strip member to the bone.

In another embodiment, the method comprises the following steps;
a) Inserting a retention member underneath the mucosa of an airway wall.
b) Attaching the retention member to a region of hard tissue or bone thus creating one or more depressions or furrows in the airway. Preferably, attachment of the retention member expands the posterior pharyngeal wall radially and increases the cross-sectional area of the airway.
c) Optionally, using a tether member to connect the retention member to one or more bone anchors.

### II. Second Preferred Embodiments

In accordance with the second preferred embodiments, the device comprises an airway support structure that is placed within or attached to an airway wall to prevent collapse leading to complete closure and blockage of airflow. The device may be referred to as a stent although it differs from typical stent structures in several important ways. Typical stents are substantially cylindrical and generally provide uniform support or radial force to maintain the patency of a luminal organ. Conversely, the device of this invention provides little or no radial support or force over one portion and substantially greater radial support about the remaining portion. Preferably, the device is implanted in the upper airway of a patient so as to prevent collapse of the lateral and posterior pharyngeal walls. Preferably, the device is implanted in the region of the pharynx called the oropharynx. The regions of the pharynx including the oropharynx are shown in FIG. 1. Preferably, the support structure is elastic to allow some movement of the pharyngeal walls without discomfort. In sleep apnea, the tongue collapses against the posterior wall of the oropharynx as shown in FIG. 2. However, complete restriction of tongue movement is not a desirable treatment approach because tongue movement is essential to swallowing and speech. The device comprises a tongue movement resisting member that readily allows movement across much of the airway but becomes progressively more resistant as it approaches the posterior pharyngeal wall or complete closure of the airway. The device is implanted with the tongue resisting member supporting the back of the tongue to provide low resistance to most tongue movement while preventing complete collapse and blockage of substantially all airflow.

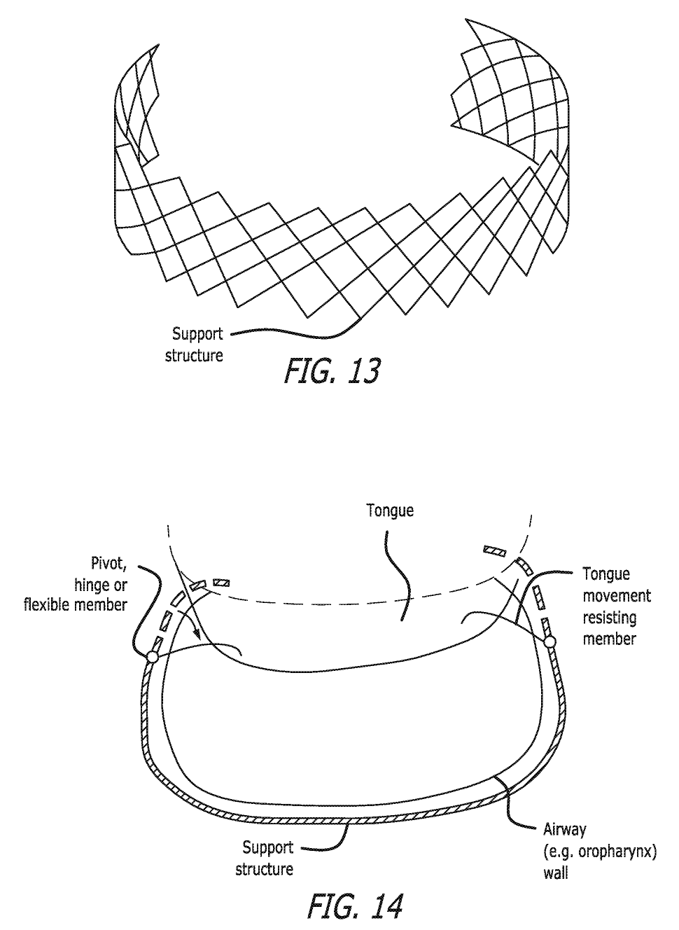

In one embodiment, the device comprises an arcuate airway support structure with a substantially semi-circular cross-section as shown in FIG. 13. An exemplary support structure is described by Pflueger et al. in U.S. Patent Application 2006/0157055 which is herein incorporated in its entirety by reference. In one embodiment, the support structure comprises a radially expandable lattice or fenestrated framework as is well known in the art of vascular stents. Preferably, the thickness of the struts of such a lattice would be less than about 0.5 mm and more preferably between about 0.1 mm and 0.25 mm. Thin struts facilitate penetration of the airway wall mucosa when the device is expanded during implantation. The device may be expanded by a radially expanding device such as a balloon or mechanical apparatus or it may be self expanding due to elasticity, shape memory or other responsive material behavior. Optionally, energy (e.g. electrocautery) may be delivered to the device to facilitate penetration of the mucosal wall during delivery.

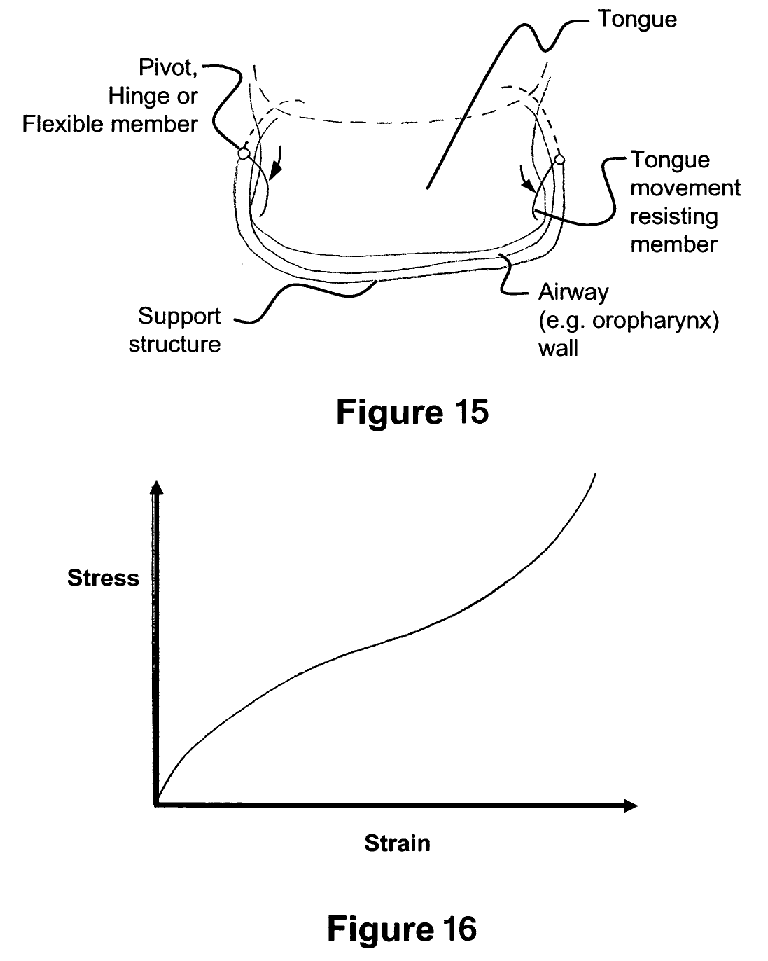

In one embodiment, the device further comprises a tongue movement resisting member. In one embodiment, the tongue resisting member comprises one or more arcuate structures. The tongue resisting structures may be integral to the support structure with a pivot, hinge or flexible element connecting them to support structure. The resisting members rotate upon posterior movement of the tongue. The pivot, hinge or flexible element allows low resistance rotation of the tongue resisting member as shown in FIG. 14. When the tongue is thrust back fully to engage the posterior pharyngeal wall, the tongue resisting members are stopped by either the support structure, pharyngeal wall or other physical stop as shown in FIG. 15. Thus, the complete collapse of the airway is prevented and at least some airflow maintained.

In one embodiment, the device comprises a tongue movement resisting member that spans the opening of the semi-circular support structure to form a closed cross-section. The tongue resisting member is preferably highly elastic and flexible thus allowing at least some tongue movement with relatively small resistance. Preferably, the tongue resisting member comprises an elastomer such as silicone or a silicone copolymer. Since elastomers typically exhibit nonlinear stress-strain behavior with a generally concave up stress-strain curve at higher strains, the modulus (and thus the resistance to further stretching) increases as the material elongation increases as shown in FIG. 16. Preferably, the tongue resisting member is constructed so that when the tongue moves to the back of the throat, the tongue resisting member material is in the region of increasing (concave up) stress-strain behavior. Therefore, the tongue resisting member will become progressively more resistant to further movement as it approaches the opposing wall (back) of the throat or posterior pharyngeal wall. Alternatively, the tongue resisting member comprises a material that promotes growth of tissue or “tissue engineering”. Biological tissue typically exhibits non-linear stress-strain behavior. Preferably, the growth promoting material is substantially replaced over time by natural tissue and is thus either bioresorbable (e.g., breaks down and is absorbed by a cell, tissue, or other mechanism within the body), bioabsorbable (similar to bioresorbable), bioerodable (e.g., erodes or degrades over time by contact with surrounding tissue fluids, through cellular activity or other physiological degradation mechanisms), biodegradable (e.g., degrades over time by enzymatic or hydrolytic action, or other mechanism in the body), or dissolvable. Each of these terms is interpreted to be interchangeable.

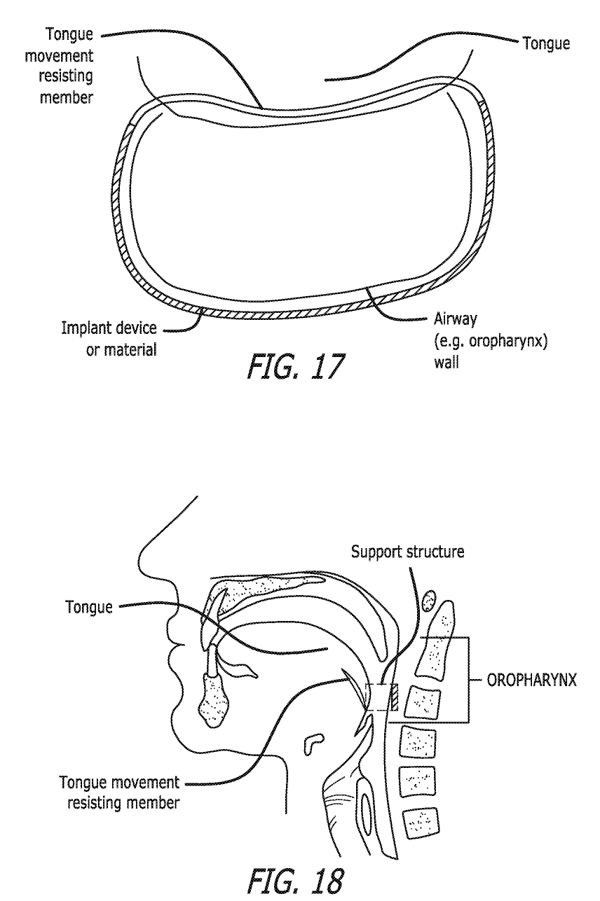

In one embodiment, the tongue movement resisting member is integral to the support member so that the device structure is a closed continuous cross-section. It may generally cylindrical but is preferably of substantially rectangular shape with rounded corners. One or more of the sides may preferentially be curved. Preferably, the side to be positioned against the tongue is concaved as shown in FIG. 17. Preferably, the flexibility of the tongue side is substantially more elastic and flexible than the other sides. Greater flexibility may be accomplished by adjustments to strut thickness, surface treatment (e.g. electro-polishing or grinding), heat treatment, coatings, density, design and combinations thereof.

In one embodiment, the device comprises a tongue movement resisting member that is attached to the support structure and extends into the tongue. A tongue movement resisting device is described by Fege in WO99/32057 which is herein incorporated in its entirety by reference. Preferably, the tongue resisting member extends substantially along or parallel to midline of the tongue. The tongue resisting member is connected to the support structure at one or more points. The tongue resisting member or the connection preferably comprises a torsion resistor such as a torsion spring to allow rotation of the tongue resisting member with tongue movement as shown in FIG. 18. Preferably, the torsion resistor has a stop or second torsion spring to provide increased resistance to tongue movement surpasses a predetermined amount. If a second torsion spring is used, preferably it has a greater spring constant than the first spring.

The device may be made of various biomaterials known in the art of implant devices including but not limited to polymers, metals, biological materials and composites thereof. Suitable polymers include acrylics, polyurethanes, silicones, polypropylene, polyvinyl alcohol, polyesters (e.g. polyethylene terephthalate or PET) and PolyEtherEther Ketone (PEEK). Suitable metals include cobalt-chrome alloys, nickel-titanium alloys (e.g. nitinol), platinum, stainless steel, titanium, gold, and tungsten. Optionally, one or more surfaces of the device may be porous (preferably microporous) to facilitate the ingrowth of tissue. Optionally, the device may comprise fibers or strands that are woven, braided or knitted as is known in the art of textile vascular grafts. Optionally, the device may be constructed to provide the elution or delivery of one or more beneficial drug(s) and/or other bioactive substances into the airway, airway wall or the surrounding tissue.

Optionally, the device may comprise an operable member that allows the device to change properties in response to change in a measured physiologic parameter. For example, a change in temperature or pressure against a portion of the device may activate a change in a physical characteristic such as bending modulus.

Also provided are methods of treating breathing disorders such as obstructive sleep apnea. In one embodiment, the method comprises the following steps:
a) Implanting a support structure in the airway of a patient, wherein the structure comprises a tongue movement resisting member that allows tongue movement for speech and swallowing with minimal resistance but more forcefully resists complete closure of the airway.
b) Preventing the complete closure of the airway by said tongue movement resisting member contacting said support structure.

In another embodiment, the method comprises the following steps;
a) Providing a support structure for implantation into the airway of a patient comprising an elastic tongue movement resisting member;
b) Implanting said structure into the oropharynx of a patient wherein the tongue resisting member increases its resistance to movement of the tongue as the tongue approaches the opposing wall of the throat.

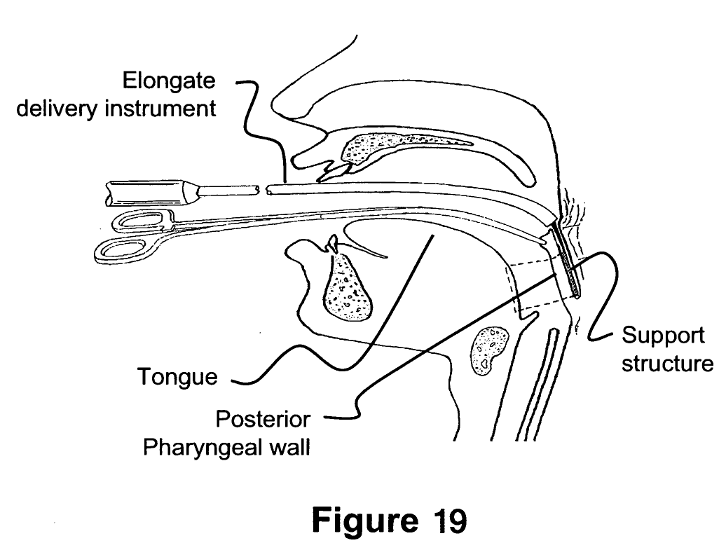

Optionally, the method may further include any of the following steps;
c) Delivering the airway support structure within or about an elongate instrument into the oropharynx via a trans-nasal or trans-oral approach as shown in FIG. 19.
d) Making an incision into the airway wall mucosa to facilitate delivery of the device into a submucosal position.
e) Radially expanding the device into the airway wall mucosa.

### III. Third Preferred Embodiments

In accordance with the third preferred embodiments, the device comprises an airway support structure that is placed within or attached to an airway wall to prevent complete collapse leading to blockage of airflow.

These devices are generally similar to a stent, although they differ from typical stent structures in several important ways. Typical stents are substantially cylindrical and generally provide uniform support or radial force to maintain the patency of a static luminal organ such as a blood vessel. They generally are designed to keep external tissue from encroaching into the lumen of the stent. Conversely, the described embodiments of this invention are can be implanted in the airway of a patient adjacent to very mobile tissue and/or structures such as the tongue. The described embodiments further allow adjacent tissue or organs to temporarily move into and even close the lumen of the structure. The described embodiments preferably include one or more regions with distinctly different radial support or force. In some embodiments, at least a portion of the device is non-cylindrical.

Preferably, a device is implanted in the upper airway of a patient. For example, a device is preferably implanted in the pharynx. In more specific examples, a device may be implanted in the nasopharynx, oropharynx, and/or hypopharynx or extend through portions of any combinations thereof. The regions of the pharynx are shown in FIG. 1.

Preferably, at least a portion or section of the support structure is elastic and radially flexible to allow movement of the tongue and/or pharyngeal walls inward into the lumen of the structure without discomfort. During swallowing, the tongue moves in the posterior direction to facilitate movement of food and fluids down the esophagus. Thus, complete restriction of tongue movement is not a desirable treatment approach because tongue movement is essential to swallowing and speech. The force exerted by the tongue during swallowing is substantially greater than when it collapses during an apneic event as shown in FIG. 2. In this example event, the force generated by the tongue is essentially due to gravity acting on the mass of the tongue.

In a preferred embodiment, the device comprises an arcuate airway support structure with a substantially semi-circular cross-section as shown in FIG. 13. An exemplary support structure is described by Pflueger et al. in U.S. Patent Application 2006/0157055 which is herein incorporated in its entirety by reference. In one embodiment, the support structure comprises a radially expandable lattice or fenestrated framework as is well known in the art of vascular stents. Preferably, the thickness of the struts of such a lattice would be less than about 0.8 mm and more preferably between about 0.1 mm and 0.25 mm. Thin struts facilitate penetration of the airway wall mucosa when the device is expanded during implantation. The device may be expanded by a radially expanding device such as a balloon or mechanical apparatus or it may be self expanding due to elasticity, shape memory or other responsive material behavior. Optionally, energy (e.g. electrocautery) may be delivered to the device to facilitate penetration of the mucosal wall during delivery.

In a preferred embodiment, the device allows movement of the tongue under typical swallowing forces but resists closure under forces normally seen during an apnea. It allows complete closure of airway under swallowing forces yet flex only relatively small amounts when the tongue relaxes. The device comprises an arcuate section and one or more flexible members. The force applied by the tongue to the posterior pharyngeal wall during swallowing is generally greater than about 2 Newtons and more often greater than about 5 Newtons. Pharyngeal pressures generated during swallowing are generally greater than 50 mmHg.¹ Conversely, during sleep the tongue relaxes and very low forces (much less than 1 Newton and generally less than 0.5N) are generated by the posterior movement of the tongue against the pharyngeal wall. Pharyngeal wall pressures generated during apnea are generally less than about 15 mmHg.² Preferably, at least a portion of the device will flex or collapse allowing the tongue to contact the posterior pharyngeal wall under a force greater than about 1.0 Newton.

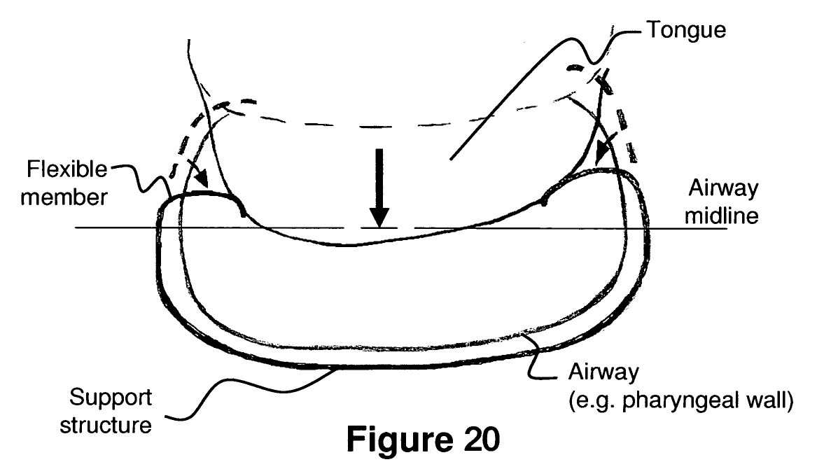

Optionally, the device may flex or collapse allowing tongue contact to the pharyngeal wall at a force between about 0.5 and 5 Newtons. Thus, the stiffness and/or design of the device will preferably require forces larger than about 0.5 Newton to cause flexure beyond the midline of the airway as shown in FIG. 20. Preferably, a force greater than about 1.0 Newton is required to flex the device to a point sufficient for the tongue to contact the posterior pharyngeal wall. Thus, in this embodiment the flex member(s) radially flex significantly, preferably greater than about 50% of the diameter or more than about 5 mm during forceful movements (e.g swallowing) of adjacent tissue or organs. However, the flex member(s) will only radially flex less than full collapse, preferably less than about 80% and more preferably less than about 40% of the diameter or less than about 4 mm under forces generated by adjacent tissue during relaxation of the muscle of adjacent tissues or organs such as occurs during an apnea.

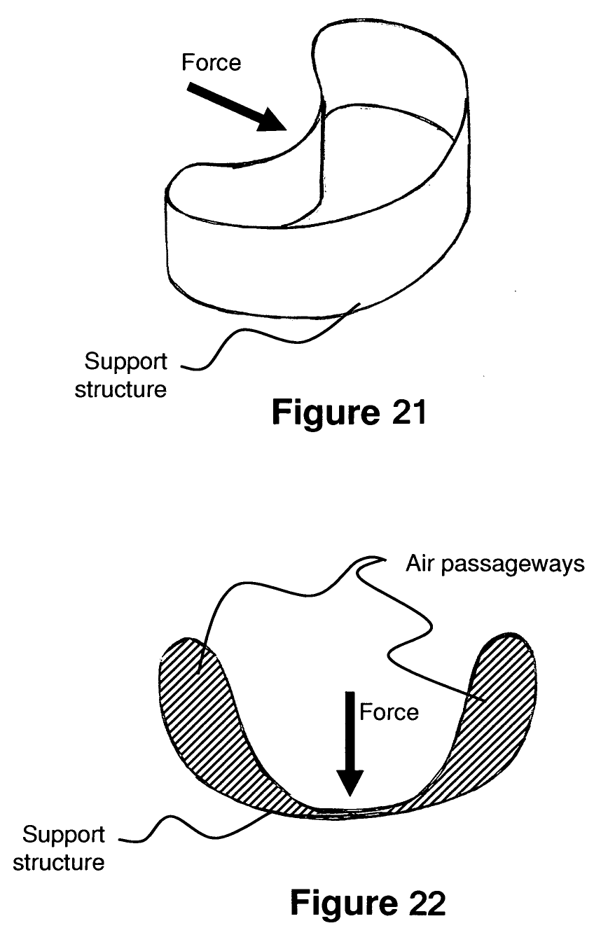

In a preferred embodiment, the device comprises a support structure with at least one flexible portion or section that is configured to allow complete posterior movement of the tongue (i.e. pressing against the posterior pharyngeal wall) while maintaining one or more airflow passages. Preferably, the device comprises at least one portion forming a closed loop or substantially cylindrical cross-section. The cylindrical portion may have a round, ovoid, or rectangular cross-section. The device may comprise a relatively rigid support portion and a relatively flexible flex portion. The more flexible portion(s) may be made of a more flexible material than the support portion and/or have struts or filaments that have substantially lower cross-sectional areas. Preferably, the cross-sectional area of the struts for filaments of the flexible portion(s) is less than 70% of the cross-sectional area of the support portion. The flex portion of the device may flex concavely (i.e. inwardly) as shown in FIG. 21.

Optionally, the region(s) of connection between the support portion and the flexible portion may comprise a transition portion. The transition portion may comprise hinge or flex elements that facilitate flexure of a portion of the support structure at one or more points. The flex elements may comprise tapered struts or wires. Preferably, the force required to flex one side of the device to opposition with the internal surface of the opposing side is more than about 0.5 Newtons and more preferably between about 1 and 5 Newtons. However, under any forces from external tissue or organs to the flex portion of the device that are within physiologic range of the upper airway, the devices flexes inward such that one or more airflow passageways are maintained as shown in the cross-section in FIG. 22.

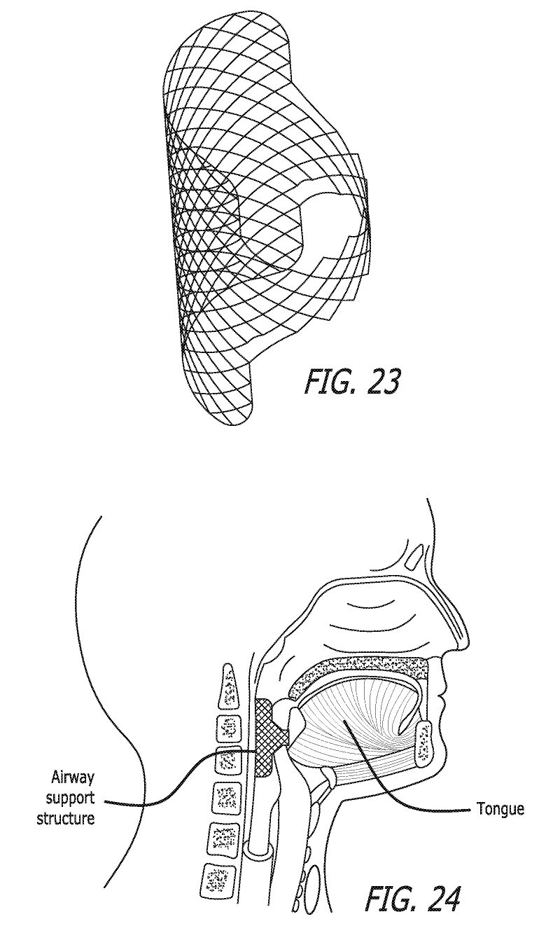

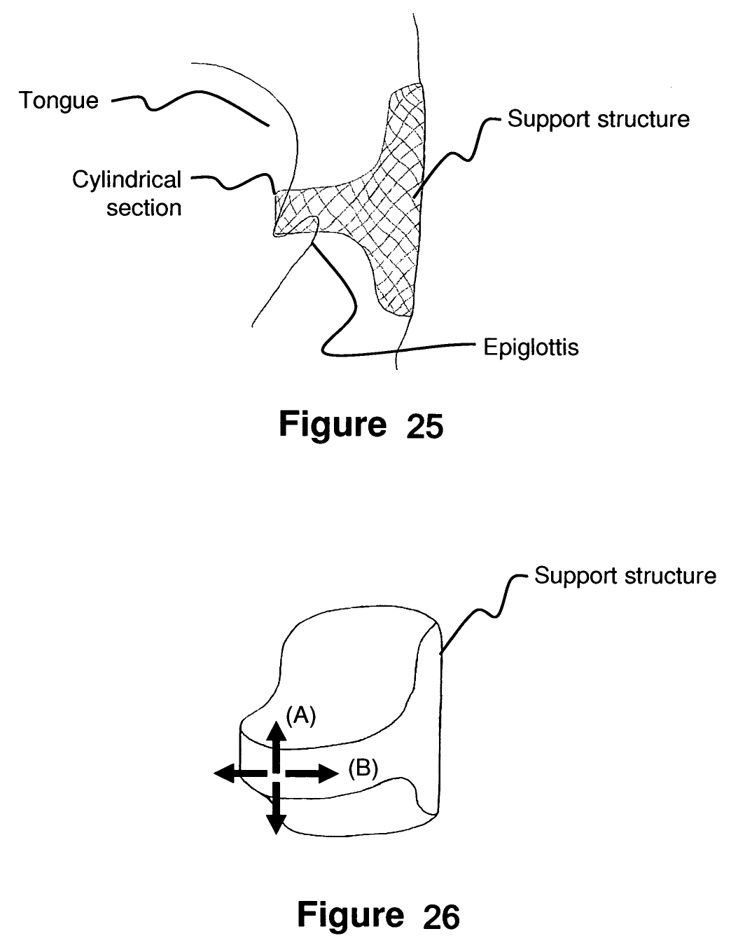

In a preferred embodiment, the device comprises an arcuate section and a cylindrical section. Preferably, the arcuate section extends longitudinally above, below and/or both above and below the cylindrical section as shown in FIG. 23. The device is implanted in upper airway and preferably in both the hypopharynx and oropharynx with the arcuate section against the posterior pharyngeal wall and the cylindrical section against the base of the tongue as shown in FIG. 24. Optionally, the cylindrical section may be anchored in the area of the upper junction of the epiglottis as shown in FIG. 25.

The flexible members and/or the cylindrical section may also have the ability to flex in a variety of non-radial directions as shown in FIG. 26. The design and/or materials may allow flexure in the longitudinal (A), transverse (B) or combinations thereof.

In one preferred embodiment, the device has a substantially rectangular-shaped cross-section where the longer sides are preferably concave. The cross-sectional shape can be concave inward, outward or both in the same direction. Preferably, both sides are concave in the same direction such that when implanted in the retroglossal (behind or near the base of the tongue) area of the pharynx, the sides are concave forward.

The device may be made of various biomaterials known in the art of implant devices including but not limited to polymers, metals, biological materials and composites thereof. Suitable polymers include acrylics, polyurethanes, silicones, polypropylene, polyvinyl alcohol, polyesters (e.g. polyethylene terephthalate or PET) and PolyEtherEther Ketone (PEEK). Suitable metals include cobalt-chrome alloys, nickel-titanium alloys (e.g. nitinol), zirconium-based alloys, platinum, stainless steel, titanium, gold, and tungsten. Optionally, one or more surfaces of the device may be porous (preferably microporous) to facilitate the ingrowth of tissue. Optionally, the device may comprise fibers or strands that are woven, braided or knitted as is known in the art of textile vascular grafts. Optionally, the device may be constructed to provide the elution or delivery of one or more beneficial drug(s) and/or other bioactive substances into the airway, airway wall or the surrounding tissue. Optionally, the device may be coated with various polymers such as a hydrogel to enhance it performance and/or biocompatibility.

In any of the previously described embodiments, the support structure may be formed at least in part of wire, ribbon, or other filamentary elements. These filamentary elements may have circular, elliptical, ovoid, square, rectangular, or triangular cross-sections. Alternatively, the support structure may be formed using conventional machining, laser cutting, electrical discharge machining (EDM) or photochemical machining (PCM). If made of a metal, it may be formed from either of either metallic tubes or sheet material. Exemplary PCM processes for making stents are described by Zadno-Azizi et al. in U.S. Pat. No. 5,907,893 and by Roth in U.S. Patent Application 2007/0031584 which are both herein incorporated in their entirety by reference.

In any of the above embodiments, the support structure may comprise one or more fixation element to facilitate fixation of the device within the airway. The fixation elements may comprise hooks, barbs, protrusions, adhesives or combinations thereof.

The device may also comprise a responsive material that allows the device to change physical properties in response to change in environmental parameter. For example, a change in temperature or pressure against a portion of the device may activate a change in a structural characteristic such as stiffness or bending modulus. In another example, the responsive material could change physical characteristics in response to the input of energy (e.g. heat or light).

Also provided are methods of treating breathing disorders such as obstructive sleep apnea. In one embodiment, the method comprises the following steps:
a) Implanting an upper airway support structure comprising a flexible member.
b) Preventing the complete closure of the airway while still allowing the tongue to move posteriorly.
c) Preventing the complete closure of the airway while still allowing the tongue to contact the wall of the pharynx.

In another embodiment, the method comprises the following steps;
a) Providing a airway support structure comprising an arcuate section and a cylindrical section.
b) Implanting said structure into the pharynx of a patient wherein the cylindrical section flexes to allow the tongue contacts wall of the throat.

Optionally, the method may further include any of the following steps;
c) Delivering the device within or about an elongate instrument into the pharynx via a trans-nasal or trans-oral approach as shown in FIG. 9.
d) Delivering the device within or about an elongate instrument comprising a radially expandable member.
e) Radially expanding said support structure into the airway wall mucosa.

In any of the above embodiments, the device may further comprise one or more sensors to measure physiologic parameters such as airway pressure. Preferably, the device further comprises a data telemetry apparatus to send the information to a receiver by wireless transmission (e.g. radio waves). The sensor device may be fabricated using micro electromechanical system (MEMS) manufacturing techniques. An exemplary wireless MEMS sensor system is described by Rich et al. in U.S. Pat. No. 6,926,670 which is herein incorporated in its entirety by reference. Alternatively, the sensor may be remotely monitored and with an external detector as described by Petersen et al. in U.S. Pat. No. 6,939,299 which is herein incorporated in its entirety by reference.

Disclosed herein is a detailed description of various illustrated embodiments of the invention. This description is not to be taken in a limiting sense, but is made merely for the purpose of illustrating the general principles of the invention. Further features and advantages of the present invention will become apparent to those of skill in the art in view of the description of embodiments disclosed, when considered together with the attached drawings and claims.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

### IV. Fourth Preferred Embodiments

A tracheotomy is an incision or opening through the front of the neck and into the trachea, or windpipe that forms a temporary or permanent opening which is called a tracheostomy. A tracheostomy creates an alternate passage called a stoma, to the lungs for air that cannot flow from the nose and mouth through the trachea because of an obstruction. Permanent tracheostomy can also be indicated for laryngeal paralysis, coma, neoplasia, severe laryngeal collapse and in cases of respiratory insufficiency due to chronic bronchitis or severe bronchial asthma.

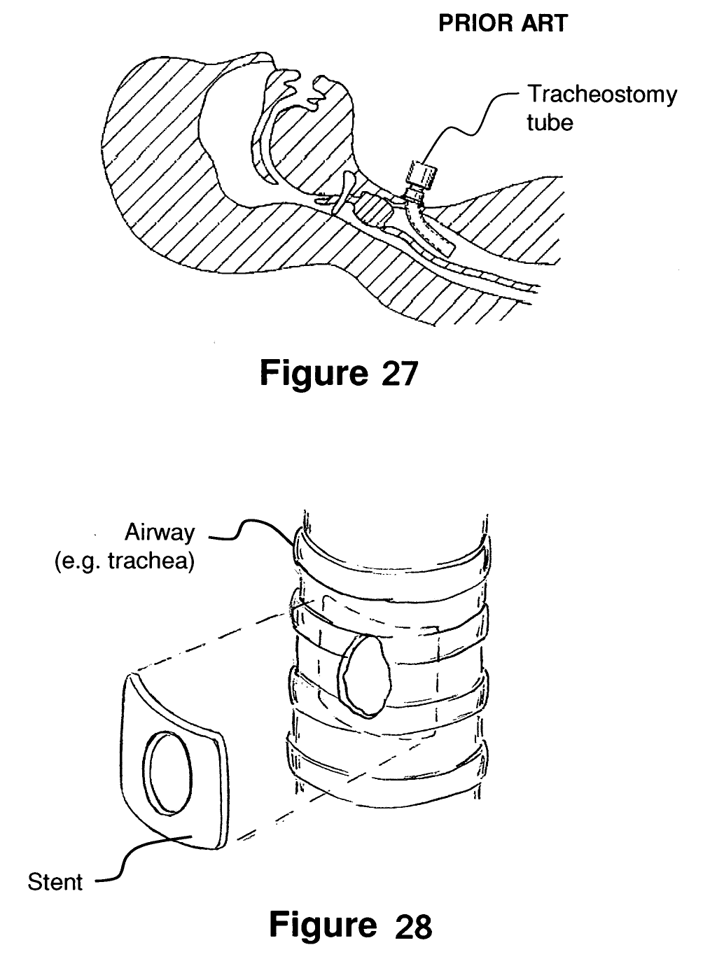

After a patient has undergone a tracheotomy, he is often provided with a tubular prosthesis, e.g., a short stationary tube, including air channel means between the trachea and the outside ambient air called a tracheostomy tube which is shown in FIG. 27. The purpose of a tracheostomy tube is to keep the airway from closing prematurely and to enable the physician to take further measures, if necessary, to ensure that the patient has a patent airway. In some cases, a prosthesis is placed in the stoma so that it may be used for long time periods, maintaining patency of the tracheostomy. Such prostheses are known as stoma stents. Patents that generally represent the state of the art in this regard include U.S. Pat. Nos. 5,107,828; 5,059,208; 5,738,095; 6,971,382; 7,021,314, each of which is hereby incorporated by reference.

Patients with an open tracheostomy stoma experience significantly compromised speech due to the associated changes in airflow patterns. In order to speak, the exhalation of air through the tracheostomy must be prevented, thus forcing air to flow through the vocal chords. Accordingly, many devices incorporate a one-way valve or check-valve on the exterior end of the tracheostomy tube to automatically close the tube during exhalation and force the air upward through the vocal chords and mouth. Unfortunately, the valve structure appears at the patient's neck which is of an unusual appearance and the patient is often self-conscious of the apparatus. This is particularly undesirable for an OSA patient who only needs the tracheostomy for sleeping. While tracheostomy has been almost uniformly effective in relieving OSA, it is used in only a small percentage of patients.

Thus, there has been a long felt need for a better treatment of breathing disorders with an unobtrusive device which performs all the necessary functions of tracheostomy tube and valve but is more acceptable to patients.

A method and apparatus are provided for the treatment airways. The device is particularly useful for the treatment of breathing disorder such as obstructive sleep apnea in patients with a tracheostomy but it may be useful for access and/or therapy for any airway procedure. The device comprises a stent structure and an operable closure apparatus. The stent structure is adapted for attachment within or about the periphery of an airway opening such as a tracheostomy and serves to maintain patency of the opening. Preferably, the stent structure is an annular structure. The stent structure may be substantially solid or a lattice. Preferably, at least a portion of the stent structure extends through the airway opening. Preferably, the stent structure forms an attachment or is attached by additional attachment members to the airway wall. The stent structure may be attached to the opening, the inner wall, the outer wall or a combination thereof. The attachment of the stent to the airway wall (e.g. trachea) may be by adhesives, friction, malleability, clamping force, sutures, hooks, barbs, staples, or other attachment means known in the art. The term stent is defined for use herein as a structure for maintaining the opening of a natural or artificial luminal organ, aperture or tissue opening.

In one embodiment, the operable closure apparatus comprises one or more closure member(s) that allows for the patient to substantially close the opening in the skin surrounding the airway opening. Preferably, there are from 2 to 4 closure members. Preferably, the closure members are attached to the inside of the skin around the airway opening. The closure members may be attached to the skin by adhesives, clamping force, sutures, hooks, barbs, staples, or other attachment means known in the art. Preferably, the device is substantially covered by skin when the closure members are closed. Alternatively, the closure apparatus may be integral to the stent structure rather than separate but attached members. In one embodiment, the integral closure member can be manipulated from a closed state to an open state. Alternatively, the closure member(s) may be a separate device and not connected to the stent.

In one embodiment, the device is implanted such that substantially all of the implant is subcutaneous. Alternatively, a portion of the closure apparatus may be exterior to the skin surrounding the airway opening. Preferably, when the operable closure apparatus is closed the device is not visible to a casual observer. Preferably, only a small slit or tissue opening is visible from the exterior. Alternatively, a small portion of the implant may be visible from the outside but is camouflaged with coloration and/or texturization so as to appear similar to the skin of the neck.

In one embodiment, the stent structure comprises a lattice such as a wire mesh, fenestrated tube or other lattice structure known in the art of stents for implant in luminal organs. Preferably, the lattice is radially expandable. Preferably, the lattice has at least one flange for attachment to the tissue adjacent to the airway opening. An exemplary wire lattice stent structure is described by Amplatz et al. in U.S. Pat. No. 6,468,303 which is herein incorporated by reference.

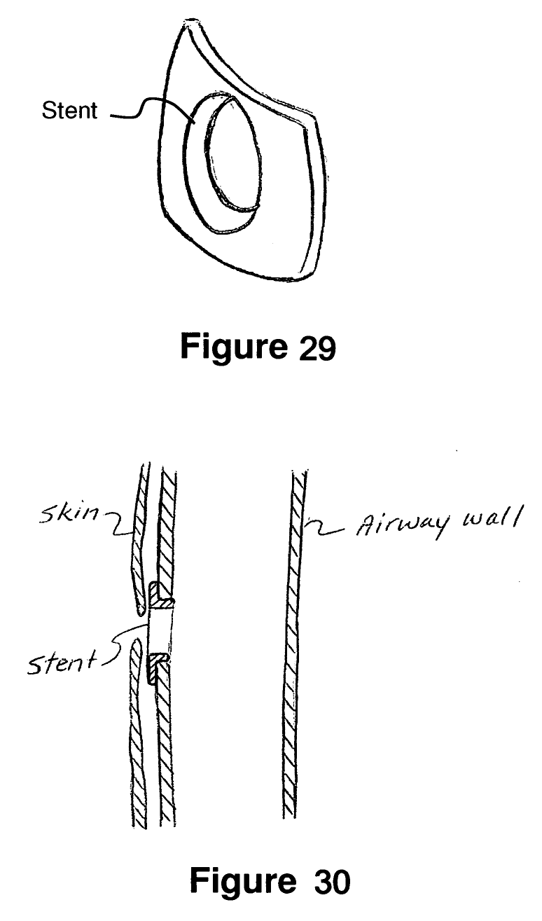

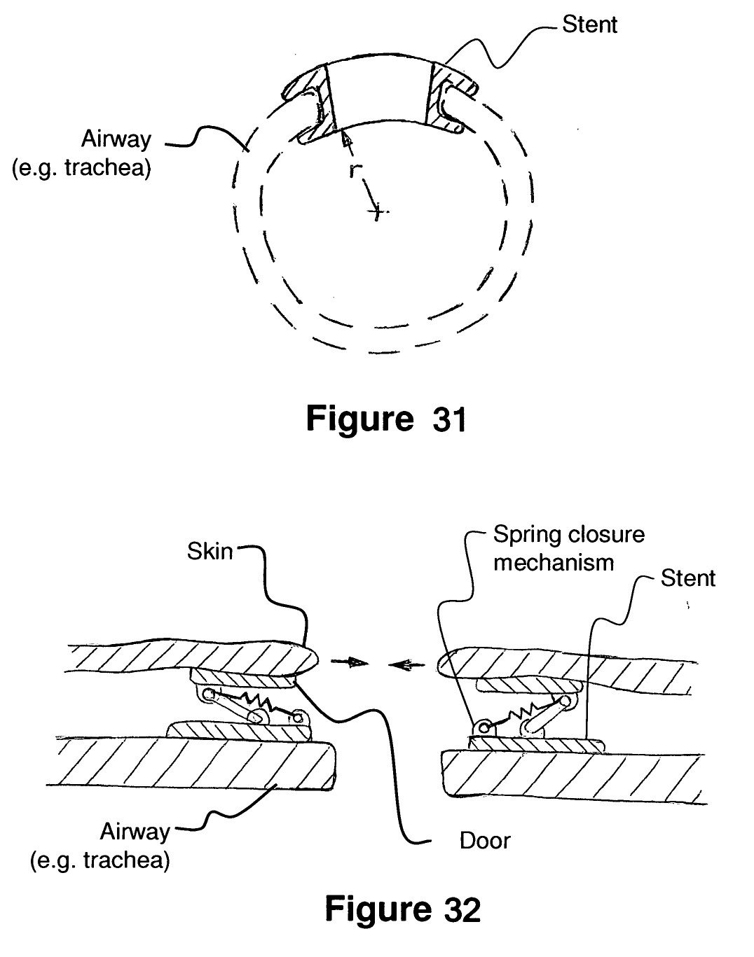

In one embodiment, the stent structure comprises a tube, disc, ring and/or flange. Exemplary structures are shown in FIGS. 28 through 31. As shown in FIG. 28, the stent may be a simple flange or disc with an aperture that is attached to the exterior surface of the airway surrounding the periphery of the airway opening. As shown in FIGS. 29, 30 and 31, the stent may comprise a tube and one or more flanges. With two flanges, a generally U-shaped cross-section is formed. Optionally, the flange(s) may be curved to generally match the curvature of the airway wall as shown in FIG. 31. Preferably, the gap between two flanges is between about 0.5 mm and 5.0 mm and more preferably between 1 mm and 3 mm.

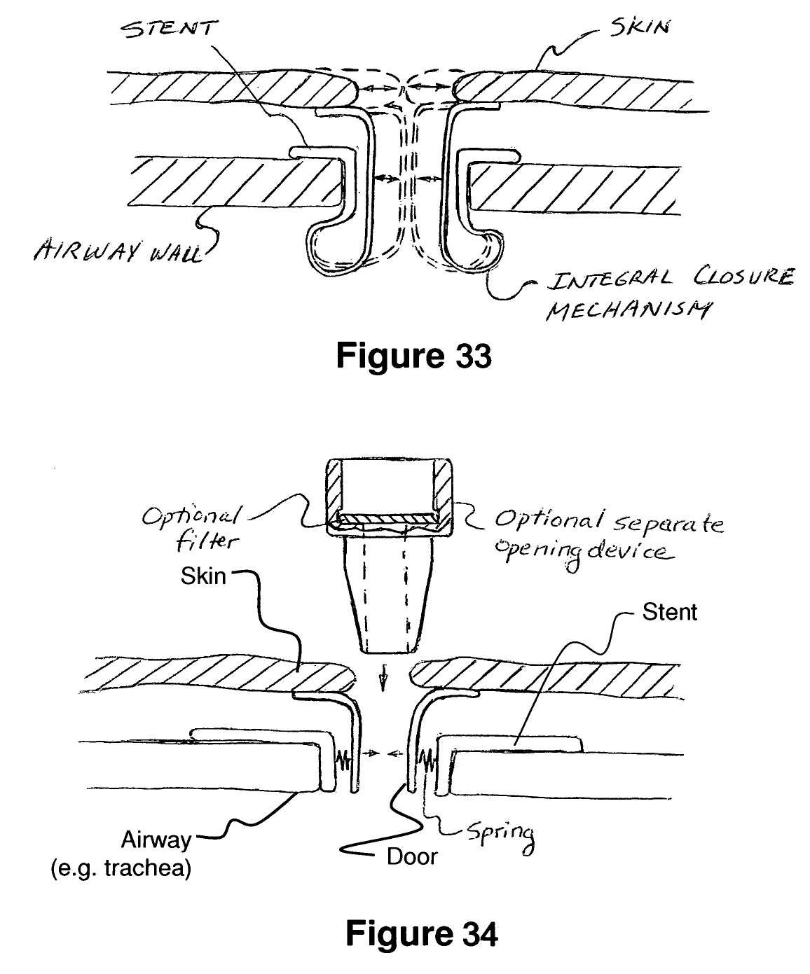

Preferably, the leaflets or doors of the closure apparatus move relative to the stent structure. The leaflets or doors may rotate, slide, bend, toggle, articulate or pivot relative to the stent. Preferably, the skin surrounding the airway opening is moved to substantially cover the airway opening when the leaflets or doors are moved to a closed position. In one embodiment, the closure member comprises two or more leaflets or doors that are operable to close the opening in the skin around an airway opening as shown in FIG. 32. A closure apparatus operates to close the doors. Preferably, the closure member(s) have a normally closed position and selectively operable to an open position. In one embodiment, the closure apparatus may be integral to the stent structure as shown in FIG. 33. Preferably, the integral closure member incorporates a spring portion or member(s) to allow for automatic closing of the opening.

Optionally, the device may comprise a flow adjustment mechanism to adjust the air intake. Optionally, the device may comprise an air flow modifier.

Optionally, the operable closure apparatus is remotely operable by a control apparatus. The remote control apparatus communicates with the closure apparatus by such means known in the art of remote control devices including but not limited to radio frequency, light, sound and magnetism.

In one embodiment, a separate opening device may be inserted into the closure apparatus to hold open closure members that are normally in a closed position as shown in FIG. 34. Alternatively, the opening device dilates the stent lumen as it is inserted. Preferably, the opening device comprises a tapered portion to facilitate insertion into and/or dilation of the closure apparatus. Preferably, the opening device temporarily attaches to the stent structure and/or closure members to stabilize the engagement. A detent or interlocking mechanism may be used to temporarily attach the opening device to the stent. The patient may insert the opening device to hold the closure member(s) open for a select period of time such as for sleeping. Optionally, the opening device may comprise a filter or valve. Upon removal of the opening device, the spring member(s) would cause the closure members to return to the closed position. Preferably, the closure members are attached to skin so that when allowed to close, they pull the skin to substantially coapt thus substantially covering the device from view.

Optionally, the device may comprise a second valve internal to the closure apparatus. The second valve may provide a more complete blockage of airflow and fluid. The second valve may be a normally closed valve such as a duck-bill valve. The second valve can be opened by insertion of a closure member opening device as described.

Optionally, the device comprises a tracheal tube that extends from the stent into the trachea. Optionally, the tracheal tube may further comprise an inflatable cuff member.

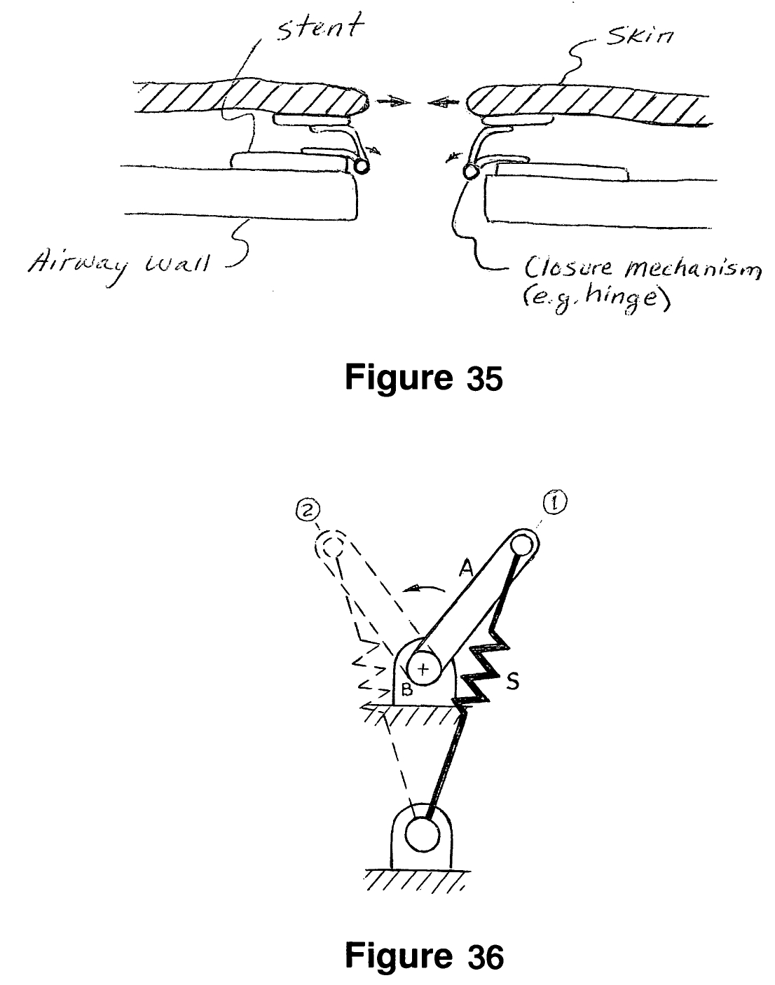

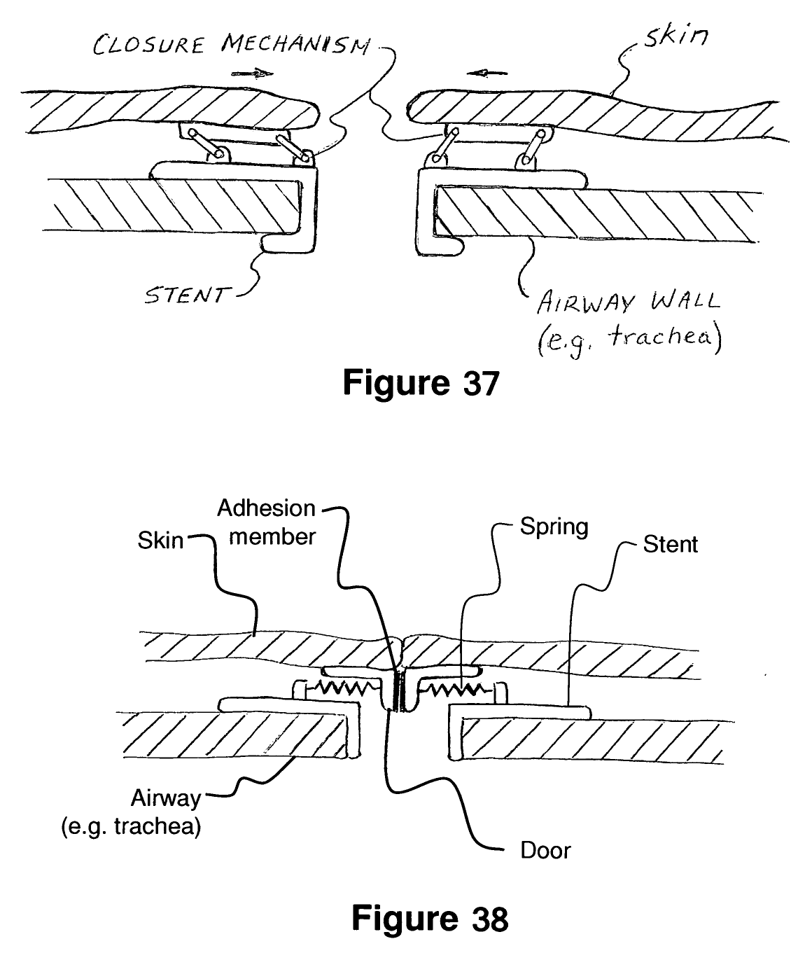

In one embodiment, the closure mechanism comprises a hinge structure that connects the leaflets to the stent as shown in FIG. 35. Optionally, the closure apparatus comprises one or more springs to facilitate opening, closure or both. In one embodiment, the closure mechanism forms an over-center spring mechanism that facilitates both opening and closure. A schematic diagram of an over-center spring mechanism is shown in FIG. 36. As shown, the spring member, S applies force to keep articulating member A at position 1. As the articulating member is rotated past its pivot point A, the force applied by the spring member changes to encourage movement toward position 2. This type of mechanism is useful for a closure mechanism that facilitates both opening and closure. FIG. 37 shows conceptually how an over-center closure mechanism could be configured in one embodiment of the invention (springs not shown for clarity).

In one embodiment, a portion of the device is removable and disposable. The removable member may be all or part of the closure apparatus. In one embodiment, the disposable portion comprises a malleable member that is easily bent.

The device may comprise a filter such as screen, mesh or that described by Bezicot is U.S. Pat. No. 5,487,382 which is herein incorporated by reference. As described, the filter may comprise an open-cell polymer foam which preferably combines appropriate hydrophilic properties to provide a heat and moisture exchanger between the exhaled and inhaled air.

The device may be fabricated using biocompatible polymers, metals, biologic materials, and/or composites thereof. Exemplary polymeric materials include polypropylene, polyvinylpyrrolidone, polyacrylics, polylactides, polyamides, polyvinyl alcohol, polyester, polyurethane, polyglycolic acid, polyfluorocarbons, hydrogels, and silicones. Exemplary metallic materials include platinum, tantalum, tungsten, gold, titanium, tin, nitinol (nickel-titanium alloy), stainless steel, Elgiloy (cobalt-chromium-nickel alloy). Exemplary biologic materials include alginates, hyaluronic acid, fibrin, collagen, silk, and small intestinal submucosa (SIS). Optionally, one or more surfaces of the device can be coated, impregnated, grafted, bound, or modified to deliver drugs, therapeutic compounds, antibacterial agents, proteins, genes, bioactive agents, growth factors or cellular material.

In any of the above embodiments, one or more portions of the device may comprise a bioadhesive to enhance the adhesion and sealing to tissue as shown in FIG. 38. The adhesive may be a coating. Any of a number of bioadhesives known in the art could be used including but not limited to cyanoacrylate based adhesives, hydrogel adhesives, alginate-based adhesives, fibrin-based adhesives, and collagen-based adhesives.

Also provided are methods of treating breathing disorders such as obstructive sleep apnea. In one embodiment, a method is provided for treating an opening in an airway. The method comprises the following steps:
a) Attaching a stent structure to at least one surface of an opening in an airway of a patient, wherein the stent structure comprises a closure apparatus that is substantially subcutaneous to skin surrounding the airway opening.
b) Operating the closure apparatus to selectively allow air flow through airway opening as needed or desired by the patient.

Optionally, the method may further include any of the following steps;
c) Attaching the stent structure to a patient's trachea using clamping force, sutures, hooks, barbs, staples and/or a bioadhesive.
d) Attaching the closure apparatus to the inside of the skin surrounding the airway opening.
e) closing the closure apparatus such that the attached skin is moved to substantially reduce the opening and/or cover the airway opening.
f) Delivering a signal (e.g. electromagnetic) to the device to affect remote operation of the closure apparatus.

Disclosed herein is a detailed description of various illustrated embodiments of the invention. This description is not to be taken in a limiting sense, but is made merely for the purpose of illustrating the general principles of the invention. Further features and advantages of the present invention will become apparent to those of skill in the art in view of the description of embodiments disclosed, when considered together with the attached drawings and claims.

1. (canceled)

2. An apparatus for maintaining the patency of an airway that is attached to an airway inner surface comprising one or more protrusions and/or air passageways, wherein the flow or air through said airway is preserved by said protrusion(s) or air passageways during posterior collapse of the tongue.

34. (canceled)

5. An apparatus for maintaining the patency of an airway that is implanted in an airway wall comprising one or more tongue movement resisting members, wherein the tongue movement resisting members become progressively more resistant as the tongue approaches the posterior pharyngeal wall.

6-8. (canceled)

9. A method for treating obstructive sleep apnea, comprising:
a) Providing an arcuate support structure defining a lumen comprising one or more flexible members, and;
b) Implanting said support structure into the airway of a patient; and;
c) wherein said flexible member(s) flex allowing adjacent tissue or organs to encroach on the lumen of said support structure while preventing the complete closure of the airway while allowing the tongue to contact the wall of the pharynx.

10. An implant device for an opening in an airway, comprising:
a) a support structure adapted for attachment at a peripheral region of the airway opening;
b) an operable closure apparatus comprising at least one closure member that moves relative the support structure; and wherein the at least one closure member is attached to skin surrounding the airway opening.

11-12. (canceled)
